(19) United States
(12) Patent Application Publication
(10) Pub. No.: US 2004/0121421 A1
(43) Pub. Date: Jun. 24, 2004

(54) Homocysteine assay adaptable to screening

(76) Inventors: Qinghong Han, San Diego, CA (US); Yuying Tan, San Diego, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/655,607

(22) Filed: Sep. 3, 2003

Related U.S. Application Data
(60) Provisional application No. 60/408,315, filed on Sep. 3, 2002.

(51) Int. Cl. C12Q 1/37

(52) U.S. Cl. 435/23

(57) ABSTRACT

A homocysteine assay that can be performed on very small quantities of blood is described. This assay is suitable for screening, including screening newborns for homocysteinuria.

### CROSS-REFERENCE TO RELATED APPLICATIONS

This application claims priority under 35 U.S.C. § 119(e) to U.S. provisional application 60/408,315 filed Sep. 3, 2002. The contents of this application are incorporated herein by reference.

### TECHNICAL FIELD

The invention is directed to a method for assessing the level of homocysteine in whole blood using very small samples. More particularly, the invention concerns an adaptation of homocysteine assays to formats which permit screening for abnormalities.

### BACKGROUND ART

The level of homocysteine (or total homocysteine, tHcy) in the blood is important as a risk factor for cardiovascular and other diseases. This is true in both adults and children. In newborns, errors in tHcy metabolism can lead to homocysteinuria as well as cardiovascular disease, mental retardation and other diseases as described by Mudd, S. H., et al., In: Scriver, C. R., et al., eds., The Metabolic and Molecular Bases of Inherited Disease, Vol. II, New York: McGraw-Hill (2002) 2007-2043. Screening methods for inborn errors of tHcy are either indirect, such as by measuring methionine, or are overly complex. See, for example, Gempel, K., et al., Clinical Chemistry (2001) 46:122-123; Chace, D. H., et al., Clinical Chemistry (2001) 86:122-123; Probst, R., et al., Clinical Chemistry (1988) 44:1567-1569.

A more straightforward assay for total homocysteine and a homocysteinase with the desired specificity have been reported by Tan, Y., et al., Clinical Chemistry (2000) 46:1686-1688 and Tan, Y., et al., Clinical Chemistry (2003) 49:1029-1030 and is also described in PCT publication WO 99/05311; PCT application US 00/17838; and U.S. Pat. Nos. 5,985,540; 5,998,191; 6,066,467; 6,140,102 and 6,468,762. The contents of these documents are incorporated herein by reference.

It has now been found possible to adapt the assay for total homocysteine described in the foregoing documents to small quantities of blood and to dried samples. By this adaptation, the assay becomes suitable for screening newborns as well as for high throughput assay formats.

### DISCLOSURE OF THE INVENTION

The invention is directed to a method to measure the level of homocysteine in small quantities of blood, which may be dried for preserving the samples until a convenient assay can be run. As little as 2-20 μl of blood may be used directly in this assay and dried on suitable solid supports for later use.

Thus, in one aspect, the invention is directed to a method to assay total homocysteine in blood, which method comprises providing a dried blood sample on a solid support; extracting said blood from the solid support in aqueous buffer; treating the extract with a homocysteinase of sufficient specificity that at least about 90% of the hydrogen sulfide produced by the action of homocysteinase upon contacting said blood sample is contributed by homocysteine when with concentrations of homocysteine and cysteine in the blood are, respectively, about 5-15 μM and about 100-300 μM respectively; and determining the level of hydrogen sulfide produced; thereby determining the level of homocysteine in the blood.

In other aspects, the invention is directed to methods to screen newborns for homocysteinuria by conducting the above assay and for detecting homocysteine metabolism abnormalities in adults and other subjects.

### BRIEF DESCRIPTION OF THE DRAWINGS

FIG. 1 shows a calibration curve for the assay of the invention.

FIG. 2 shows the correlation of the level of tHcy determined in plasma and by use of the method of the invention.

### MODES OF CARRYING THE INVENTION

The invention provides an adaptation of assays for homocysteine, such as those described in the above-referenced documents. Briefly, the assay takes advantage of homocysteinase enzymes that are sufficiently specific that the hydrogen sulfide generated by treating biological fluids with this enzyme is contributed almost entirely by homocysteine, and interference from cysteine is minimized. In general, such enzymes are sufficiently specific that at least about 90% of the hydrogen sulfide produced when contacted with a biological sample is due to homocysteine, even when homocysteine is present only at about 5 micromolar and cysteine is present at about 300 micromolar.

Thus, the capability of the assay to be performed on dried blood samples and on samples of very low volume is dependent on a combination of factors. One factor is the ability to extract the components of dried blood preserved on a solid support so as to solubilize the homocystine and homocysteine in the sample. Typically, a reducing agent is added to the extracting solution in order to convert any homocystine present to homocysteine. An anticoagulant, such as heparin or EDTA, may also be added. Second, because the assay measures the amount of hydrogen sulfide Produced by treating the sample with homocysteinase, any interference from methionine, which methionine is always present in blood, is eliminated. Any lysis of any methionine present yields ammonia, α-ketoglutarate, and methylmercaptan, not hydrogen sulfide. Thus, no complex steps to account for interference from methionine are required. Indeed, tests employing the method of invention have demonstrated that even in the presence of 200 μM L-methionine, the variation in the concentration determined for total homocysteine present in the range of 15 μM per liter is <1%.

Third, because the homocysteinase used in the assay is specific for homocysteine as compared to cysteine, even addition of 200 μM cysteine to the assay for total homocysteine in the range of 15 μM has <2% effect on the result. Typically, in normal individuals, the level of homocysteine is in the range of 5-15 μM.

Because of this combination of these factors, extremely small quantities of blood may be dried onto solid supports, extracted and assayed. Such assays would not be possible were it necessary to perform multiple steps. Multiple steps are not necessary in with assays of the present invention.

Thus, in general, the assay comprises the following steps, all performed without sample separation:

1. Reduction of any homocystine present to homocysteine—this can be accomplished in the course of the extraction step if desired.

2. Conversion of L-homocysteine to the reaction products α-ketobutyrate, ammonia and hydrogen sulfide.

3. Detection of hydrogen sulfide.

Various methods for detecting and measuring the level of hydrogen sulfide could be used. For example, hydrogen sulfide reacts with lead ion in solution to form a black precipitate; it would be possible to read the intensity of opaqueness of the resulting precipitate. As described in the above referenced patent application, a very convenient method employs generation of color or fluorescence by adding development reagents which include an oxidizing agent, such as ferric ion and a color generating reagent such as N,N-dialkyl phenylene diamine. Using such reagents, either the color developed can be measured or the fluorescence generated upon excitation can be used as a measure of the hydrogen sulfide generated. An outline of this method wherein H₂S combines with N,N-dibutyl phenylene diamine chloride (DBPDA) to form a colored thiazine which can be detected quantitatively at OD₆₆₀ nm is shown below:

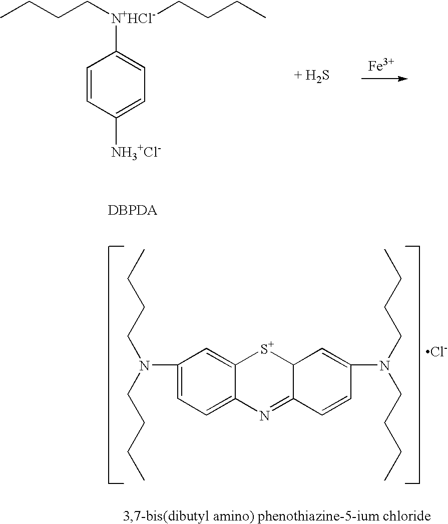

These systems for detection are described in the above referenced documents.

It has now been found that this general type of assay is adaptable to very small samples of blood which need not be separated into plasma or serum and which may be preserved for assay by drying the blood onto a solid support. For example, the sample is most conveniently dried on a filter paper or other suitable support such as nylon or other fabric. Filter paper is probably the most convenient.

By use of the method of the invention, very small samples of blood can be used, typically no more than 20 microliters, preferably no more than 5 or 10 microliters and preferably no more than 1 microliter. Because no separation is required, this sample size is readily manipulated. As stated above, it is desirable to treat the sample with a reducing agent to produce homocysteine from any homocystine. Conveniently, the reducing agent can be added to the extraction medium. It is also desirable to add an anticoagulant, such as heparin or EDTA, to the extraction medium.

In view of the small sample size and the convenience of handling, the methods of the invention are appropriate for large scale screening, including the screening of infants for evidence of homocysteinuria. The availability of this assay permits adjustments to be made to the diet and treatment of the infants in time to prevent the undesirable sequelae of this condition. The assay can, of course, also be used on adults and children and can thus form a convenient on-site assay for abnormal levels of homocysteine. Nevertheless, because the assay method is adaptable to routine screening of newborns, it can be added to the repertoire of tests used to identify conditions which can be treated when recognized early.

The assay method of the invention has the further advantage that the amounts of blood components normally present do not significantly interfere. It has been demonstrated that protein concentrations up to 20 mg/ml show <4% interference; hemoglobin concentrations of up to 1 mg/ml show <10% interference; lipid concentrations of up to 5 mg/ml show <10% interference; bilirubin C and bilirubin F concentrations up to 0.4 mg/ml show <10% interference. Thus, even in the case of elevated bilirubin levels associated with jaundice, the assay of the invention provides accurate results.

As stated above, the homocysteine levels found using the tests are compared to those in the normal range (5-15 μM) and elevated levels above the normal range signify the necessity for correction.

The following example is intended to illustrate but not to limit the invention.

### EXAMPLE 1

### Determination of Homocysteine in Whole Blood

Five μl of whole blood is spotted in duplicate onto filter paper and dried at room temperature for at least 3 h.

To prepare the whole blood spot samples for assay, the entire spot is cut from the filter paper and transferred to a conical microcentrifuge tube.

The spot is incubated at 37° C. for 30 minutes in 0.5 ml of phosphate-buffered saline, containing 1.0 mM DDT (reducing agent); 1.0 mM EDTA and 0.2% Tween-X100. The sample is vortexed until blood is completely extracted from the paper.

The sample solution is transferred to 1.5 ml MSI UltraFuge Centrifuge Filters (30 KD cutoff), then centrifuged at 4,000 g for 10 minutes and transferred to another tube. 0.5 ml assay buffer, pH 8.3, is then added.

20 μl of recombinant homocysteine α, γ-lyase (homocysteinase) (0.145 mg/ml rHCYase) reagent is added and incubated at 37° C. for 3 minutes to release H₂S.

The H₂S chromophore reagent (DBPDA) is added. This reagent is in two parts: reagent 1 is 33.25 mg of potassium ferricyanate dissolved in a solution of 10 ml of 1 N NaCl, 1% (w/v) Triton x-100 to obtain a 10 mM stock solution of ferric ion: a second reagent is 52.5 mg of N, N-dibutyphenylenediamine dissolved in double-distilled water to a final concentration of 100 mM. The two reagents are added in a ratio of 20:1. Fluorescence is measured at EX640/EM675.

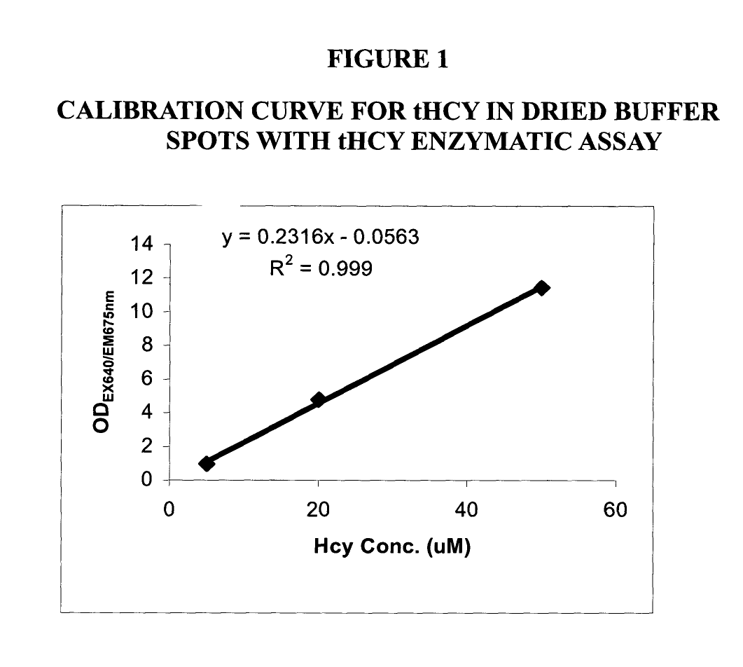

Aliquots of 10 μl of concentrations of standard solutions of Hcy at 5,20 and 50 uM were spotted onto filter paper. The standard curve had a linearity of Y=0.231X−0.0563 (R²=0.999). (FIG. 1)

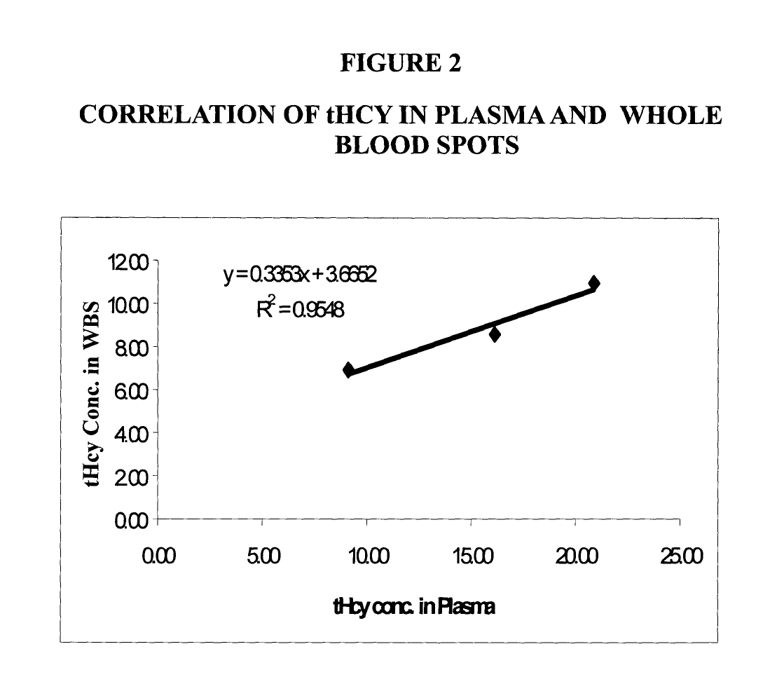

Measurements of tHcy from dried blood spots correlated well with tHcy measurements of those from plasma. Y=0.335X+3.6652 (R²⁼0.9548 n=3). (FIG. 2). Both samples were derived from the same experimental mouse.

1. A method to assay total homocysteine in blood, which method comprises
providing a dried blood sample on a solid support;
extracting said blood from the solid support in aqueous buffer;
treating the extract with a homocysteinase of sufficient specificity that at least about 90% of the hydrogen sulfide produced by the action of homocysteinase upon contacting said blood sample is contributed by homocysteine when with concentrations of homocysteine and cysteine in the blood are about 5-15 μM and about 100-300 μM, respectively; and
determining the level of hydrogen sulfide produced;
thereby determining the level of homocysteine in the blood.

2. The method of claim 1, which includes the step of adding reducing agent to the extract.

3. The method of claim 2, wherein said adding is to the aqueous buffer during the extracting.

4. The method of claim 1, wherein said aqueous buffer further contains an anticoagulant.

5. The method of claim 4, wherein the anticoagulant is EDTA or heparin.

6. The method of claim 1, wherein said determining the level of hydrogen sulfide produced is by adding an oxidizing agent and a color generating reagent.

7. The method of claim 6, wherein the color generating reagent is an N,N-dialkyl phenylene diamine.

8. The method of claim 6, wherein said determining is measured by absorbance or fluorescence of the color generated.

9. The method of claim 1, wherein the blood sample is 10 μl or less.

10. The method of claim 9, wherein the blood sample is 5 μl or less.

11. The method of claim 10, wherein said blood sample is 2 μl or less.

12. The method of claim 1, wherein the solid support is filter paper.

13. A method to screen a newborn for homocysteinuria which comprises obtaining a blood sample from a newborn and conducting method of claim 1.

14. A method to test a subject for homocysteine abnormalities in a subject which comprises obtaining a blood sample from a subject and conducting method of claim 1.
